SEQUENCE LISTING (22 sequences)

SEQ ID NO: 1 - protein, 123 aa, Homo sapiens
QVQLVQSGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 2 - protein, 123 aa, Homo sapiens
EVQLVESGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 3 - protein, 5 aa, Homo sapiens
AYYIH

SEQ ID NO: 4 - protein, 17 aa, Homo sapiens
VINPSTGTTFYAQNFPD

SEQ ID NO: 5 - protein, 14 aa, Homo sapiens
AISEHGSGSYSPYY

SEQ ID NO: 6 - protein, 107 aa, Homo sapiens
SYELTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 7 - protein, 107 aa, Homo sapiens
SYVLTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 8 - protein, 11 aa, Homo sapiens
SGEALPKKYGY

SEQ ID NO: 9 - protein, 7 aa, Homo sapiens
RDVERPS

SEQ ID NO: 10 - protein, 10 aa, Homo sapiens
LSADSSGTWV

SEQ ID NO: 11 - protein, 12 aa, Artificial Sequence
  Peptide comprising epitope of NI-204.B and NI-204.O
GGPKDEERHVGD

SEQ ID NO: 12 - protein, 11 aa, Artificial Sequence
  Peptide comprising the epitope of NI-204.B
GGPKDEERHVG

SEQ ID NO: 13 - protein, 9 aa, Artificial Sequence
  Peptide comprising the epitope of NI-204.O
KDEERHVGD

SEQ ID NO: 14 - protein, 15 aa, Artificial Sequence
  Peptide comprising the epitope of NI-204.G
HEKADDLGKGGNEES

SEQ ID NO: 15 - protein, 121 aa, Homo sapiens
QVQLVESGGGVVQPGRSLRLSCAASGYIFSSFGMHWVRQTPGKGLEWVALIWYDGSRQSYADSVRGRFTISRDNSKNTVFLQMNSLRGEDTAVYHCARTGYDDKRGGFDTWGQGTMVTVSS

SEQ ID NO: 16 - protein, 5 aa, Homo sapiens
SFGMH

SEQ ID NO: 17 - protein, 17 aa, Homo sapiens
LIWYDGSRQSYADSVRG

SEQ ID NO: 18 - protein, 12 aa, Homo sapiens
TGYDDKRGGFDT

SEQ ID NO: 19 - protein, 107 aa, Homo sapiens
SYELTQPPSVSVSPGQTARITCSGDALAKQYSYWYQHKPGQAPVMVMYKDRERPSGIPERFSGSSSGTTVTLTISAVQAEDEADYYCQSTGTDSPYIFGTGTKVTVL

SEQ ID NO: 20 - protein, 11 aa, Homo sapiens
SGDALAKQYSY

SEQ ID NO: 21 - protein, 7 aa, Homo sapiens
KDRERPS

SEQ ID NO: 22 - protein, 10 aa, Homo sapiens
QSTGTDSPYI

(19) United States
(12) Patent Application Publication
(10) Pub. No.: US 2023/0035402 A1
(43) Pub. Date: Feb. 2, 2023

(54) MISFOLDED SOD1 ASSAY

(71) Applicants: AL-S Pharma AG, Schlieren (CH); Neurimmune AG, Schlieren (CH)

(72) Inventors: Marcel MAIER, Zürich (CH); Michael SALZMANN, Zürich (CH); Jan GRIMM, Dübendorf (CH)

(21) Appl. No.: 17/912,166

(22) PCT Filed: Mar. 18, 2021
(86) PCT No.: PCT/EP2021/056933

§ 371 Date: Sep. 16, 2022

(30) Foreign Application Priority Data
Mar. 18, 2020 (EP) 20163909.3

(51) Int. Cl. G01N 33/573; G01N 33/68; G01N 33/543

(52) U.S. Cl. CPC G01N 33/573; G01N 33/6878; G01N 33/54326; G01N 2333/90283; G01N 2800/285

(57) ABSTRACT

Provided is a novel high sensitive method for assaying misfolded SOD1 in a body fluid of a subject, in particular in the cerebrospinal fluid. This method is based on a novel highly sensitive immunoassay making use of a unique epitope of SOD1 and corresponding anti-SOD1 antibodies. In addition, kits comprising the components of the immunoassay are provided.

### FIELD OF THE INVENTION

The present invention generally relates to a novel highly sensitive method of assaying misfolded SOD1 (mSOD1) in a body fluid, in particular in cerebrospinal fluid (CSF) of a subject and provides a high-sensitive immunoassay for mSOD1 as well as a kit for use in such assay.

### BACKGROUND OF THE INVENTION

Amyotrophic lateral sclerosis (ALS) is a highly heterogeneous disease with no effective treatment. This also because the causes of ALS are largely unknown, with ˜90% of cases being sporadic (sALS) while only ˜10% are familial ALS (fALS). Intensive research since the 1990's has aimed to unravel the mechanisms involved in motor neuron degeneration. These studies suggest that ALS is a complex disease driven by a combination of several systemic parameters. To date, up to 30 genes are described as monogenic causes of ALS, with the most frequent ones being C9orf72, SOD1, FUS, and TARDBP/TDP43; see for review Vijayakumar et al., Front. Neurol. 10 (2019):400. doi: 10.3389/fneur.2019.00400. While genetic linkage and thus genetic markers may be feasible for the prognosis of the susceptibility and determining correlation to fALS, assessment of sALS as well as drug development has been hampered by the lack of biomarkers that aid in early diagnosis, demonstrate target engagement, monitor disease progression, and/or can serve as surrogate endpoints to assess the efficacy of treatments. Fluid-based biomarkers may potentially address these issues. An ideal biomarker should exhibit high specificity and sensitivity for distinguishing ALS from control (appropriate disease mimics and other neurologic diseases) populations and monitor disease progression within individual patients. Significant progress has been made using cerebrospinal fluid, serum, and plasma in the search for ALS biomarkers, with urine and saliva biomarkers are still in earlier stages of development. A few of these biomarker candidates have demonstrated use in patient stratification, predicting disease course (fast vs. slow progression) and severity, or have been used in preclinical and clinical applications. However, while ALS biomarker discovery has seen tremendous advancements in the last decade, validating biomarkers and moving them towards the clinic remains more elusive; see for review Vu and Bowser, Neurotherapeutics 14 (2017), The solution to this technical problem is provided by the embodiments as characterized in the claims and disclosed further below in the description.

### SUMMARY OF THE INVENTION

The present invention generally relates to a novel highly sensitive method of determining the presence and level, respectively, of misfolded SOD1 (mSOD1) in a body fluid from a subject using an immunoassay comprising an anti-SOD1 antibody as capture antibody, which is directed to a specific epitope of SOD1. As illustrated in the appended Examples and Figures the detection of mSOD1 with the method of the present invention or assaying an increased level of mSOD1 in comparison to a control sample is indicative for ALS. More specifically, the assay of the present invention is capable of identifying with a high degree of certainty patients with sALS, which hitherto was hardly possible.

The present invention is based on the unexpected finding that a specific epitope of SOD1 and therapeutic anti-SOD1 antibodies directed thereto, respectively, are also of particular diagnostic value in the detection of mSOD1 in sample of a body fluid from a subject suspected to suffer from or being at risk to develop ALS and ALS patients, with the potential to even detect and/or differentiate between fALS and sALS.

Human Cu/Zn-superoxide dismutase (SOD1) is a 32 kDa homodimeric metalloenzyme, with the gene locus on the chromosome 21, localized predominantly in the cytosol, nucleus and peroxisomes but also in the mitochondrial intermembrane space of eukaryotic cells. It contains an active site that binds a catalytic copper ion and a structural zinc ion. The functional role of SOD1 is to act as an antioxidant enzyme catalyzing the dismutation of superoxide radical to dioxygen and hydrogen peroxide lowering in that way the steady-state concentration of superoxide and the oxidative stress to the cell (Fridovich, Science 201 (1978), 875-879).

Mutations in the gene encoding SOD1 account for approximately 20% of familial amyotrophic lateral sclerosis (fALS) cases and for a small percentage of sporadic ALS (sALS) cases (Rosen et al., Nature 362 (1993), 59-62; Chio et al., Neurology 70 (2008), 533-537; Kwon et al., Neurobiol. Aging 33 (2012), e1017-1023). ALS is a rapidly progressive, invariably fatal neurological disease that attacks the neurons responsible for controlling voluntary muscles, specifically motor neurons in the spinal cord, brain stem, and motor cortex (Bruijn et al., Annu. Rev. Neurosci. 27 (2004), 723-749). The mechanism how mutations in SOD1 lead to ALS is not fully understood, but there is evidence for a toxic gain-of-function mechanism where mutation-induced misfolding of SOD1 is associated with toxicity causing degeneration of motor neurons (Julien, Cell 104 (2001), 581-591).

However, it is also believed that misfolding of wild type (wt) SOD1 is associated with the majority of the sALS cases (Bosco et al., Nature Neuroscience 13 (2010), 1396-1403). Wildtype SOD1 is a subject of massive post-translational modifications, such as subunit dimerization, building of the intrasubunit disulfide bond between residues Cys57 and Cys146, and the coordination of copper and zinc. Disruptions of these processes have all been shown to cause wt SOD1 to aggregate (Durazo et al., J. Biol. Chem. 277 (2009), 15923-15931; Estévez et al., Science 286 (1999), 2498-2500; Rakhit et al., J. Biol. Chem. 279 (2004), 15499-15504; Lindberg et al., Proc. Natl. Acad. Sci. USA 101 (2004), 15893-15898) providing therefore a possible pathogenic model for spontaneous ALS forms. Abnormal changes of wt SOD1 have also been reported in other neurodegenerative diseases such as Alzheimer's Disease (AD) and Parkinson's Disease (PD).

While SOD1 had been considered as a potential biomarker conflicting results have been reported. For example, Jacobsson et al., Brain 124 (2001), 1461-1466, determined amounts, activity and molecular forms of SOD1 in CSF from ALS patients carrying the D90A and other SOD1 mutations and patients without such mutations, and they found no differences in amount of protein and enzymatic activities of SOD1 between 37 neurological controls, 54 sporadic and 12 familial ALS cases, and 10 cases homozygous for the D90A mutation. Likewise, as summarized in Vu and Bowser, (2017), supra, another study measured CSF SOD1 levels between patients with ALS and neurologic disease controls and failed to find significant differences between the groups and indicated that SOD1 CSF level is not a diagnostic biomarker for ALS. In addition, a later study of analysis of CSF from ALS patients and controls via ELISA assays using antibodies reacting with different sequence segments of mSOD1 species showed no significant differences between the ALS patients and the controls (Zetterström et al., J. Neurochem. 117 (2011), 91-99). The authors assumed that the estimated concentration of mSOD1 in the interstitium of the CNS is a 1000 times lower than that required for appreciable cytotoxicity in model systems. Accordingly, Zetterström et al. concluded that these results argue against a direct cytotoxic role of extracellular mSOD1 in ALS and that therefore mSOD1 in CSF cannot be used as a biomarker of ALS in patients with and without mutations in the enzyme.

Recently, Tokuda et al. (Tokuda et al. Molecular Neurodegeneration (2019) 14:42 https://doi.org/10.1186/s13024-019-0341-5) described an ELISA assay for misfolded wild-type SOD1 in cerebrospinal fluid of sALS. However, the capture antibody that was used, antibody C4F6 which is a monoclonal antibody that has been generated by using recombinant SOD1 with G93A mutation was reported to show strong immunoreactivity to denatured G93A, but much lower reactivity to other hSOD1 mutants, and very low reactivity to denatured WT hSOD1. Furthermore, the C4F6 antibody has been described to stain spinal cord tissue from A4V fALS case but not in the sALS cases; see for characterization of antibody C4F6 by Ayers et al. Acta Neuropathologica Communications 2014, 2:55 Page 2 of 13 http://www.actaneurocomms.org/content/2/1/55. Therefore, it still remains to be shown whether antibody C4F6 and the ELISA assay described in Tokuda et al. (2019) is reliable and suitable to be developed to the clinics.

In contrast, unbiased experiments performed within the scope of the present invention revealed that among a subset of different blinded anti-mSOD1 antibodies with all high but different binding affinity to mSOD1 and covering different epitopes, only two antibodies, designated NI-204.B and NI-204.O have been found to reliably detect mSOD1 in tissue, cell and body fluid samples, but not other candidates among some of which displayed considerable lower EC₅₀ values for denatured, oxidized and recombinant SOD1 as determined in conventional ELISA assays, and thus would have been first choice for use as a capture antibody in an immunoassay for mSOD1.

Decoding of the antibody probes revealed that NI-204.B and NI-204.O share a similar epitope of SOD1 within the amino acid sequence 73-GGPKDEERHVGD-84 set forth in SEQ ID NO: 11. When investigating an immunoassay for the detection in accordance with the present invention, a sandwich ELISA wherein antibody NI-204.B and NI-204.O, respectively, served a as capture antibody could be established and found to reliably detect mSOD1 in CSF samples from ALS patients illustrated in the Examples for antibody NI-204.B and NI-204.O. In principle, the pre-assays for identifying a suitable capture antibody and subsequent immunoassay are based on the assay for mSOD1 described in Gill et al., Sci. Rep. 9 (2019), 6724, https://doi.org/10.1038/s41598-019-43164-z, see “Methods” section with additional modifications for the detection of mSOD1 in body fluids such as CSF illustrated in the Examples.

Notably, in contrast to the antibody C4F6 used in Tokuda et al. (2019), the epitope recognized by antibodies NI-204.B and NI-204.O is not necessarily associated with a mutant SOD1 protein and recognized on spinal cord tissue not only from A4V fALS patients but also from sALS as well as fALS patients carrying C9ORF72 hexanucleotide repeat expansions or unknown genetic mutations; see Maier et al., Sci. Transl. Med. 10, eaah3924 (2018) 5.

Therefore, it prudent to expect that the assay of the present invention has applicability to a broader range of ALS patients than the ELISA assay described in Tokuda et al. (2019), if it works at all.

Thus, the present invention generally relates to a novel method of assaying mSOD1 in a body fluid of a human subject using an immunoassay. In particular, said immunoassay is an ELISA assay and the body fluid, preferably CSF is contacted with a first anti-SOD1 antibody as a capture antibody and a second anti-SOD1 antibody as a detection antibody

This innovative assay is of particular interest since both fALS as well as sALS can be diagnosed via assaying mSOD1 in the body fluid of a patient, wherein mSOD1 serves as a biomarker. This is important since while most of the incidents of fALS may be determined by genetic markers, identification of patients with sALS is much more difficult. Accordingly, with the new immunoassay patients with ALS, in particular sporadic ALS can be identified and selected for the treatment with anti-SOD1 antibodies and/or with other drugs currently used in the treatment of ALS and its symptoms, respectively.

The assay of the present invention can also be used to monitor the pharmacodynamic changes in the level of mSOD1 in a body fluid, preferably in CSF which can aid in the dose optimization of therapeutic agents useful in the treatment or in the amelioration of symptoms of a patient having ALS. Assays that are sensitive enough to allow accurate and precise quantification of low concentrations of mSOD1 in clinical trials of candidate therapeutics would benefit ALS research efforts.

### BRIEF DESCRIPTION OF THE DRAWINGS

FIG. 1: Detection of mSOD1 in samples of a body fluid from ALS patients. For the quantitative measurement of mSOD1 in CSF, the Ciraplex™ Human Ultrasensitive mSOD1 1-plex immunoassay kit manufactured by Aushon BioSystems was used which is a singleplex sandwich ELISA. Samples from 10 fALS patients (FIG. 1A), 6 sALS patients (FIG. 1B) and 10 non-neurological control participants (FIG. 1C) have been analyzed and the results are shown in the bar charts (FIGS. 1D and E); *p<0.05 (chi-square test of misSOD1 positive/negative cases; or Kruskal-Wallis/Dunn's multiple comparison test).

### DETAILED DESCRIPTION OF THE INVENTION

The present invention relates to a novel highly sensitive immunoassay for detecting mSOD1 in a body fluid of a subject, wherein the assay makes use of an antibody specifically recognizing an epitope disposed on mSOD1 aggregates and the assay is capable of discriminating subjects which suffer from or are at risk to develop ALS from healthy volunteers. More specifically, the present invention relates to the embodiments as characterized in the claims, disclosed in the description and illustrated in the Example and Figure further below.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-0-19852917-0.

The term “assaying” mSOD1 as used throughout the specification includes determining or measuring the presence/amount/level/concentration of mSOD1 as well as quantifying mSOD1 and related expressions.

Furthermore, unless stated otherwise, terms and expressions used herein in order to characterize the present invention are given in the definitions as provided in WO 2012/080518 A1, in particular in subsection “I. Definitions” at pages 10 to 30, the disclosure content of which is explicitly incorporated herein by reference. The same applies to the general embodiments disclosed in WO 2012/080518 A1 for antibodies, etc.

As known in the art, different neurodegenerative diseases show occurrence of or are related to mSOD1, e.g., ALS, Alzheimer's Disease (AD), ALS/parkinsonism-dementia complex (ALS-PDC), Down's syndrome and Parkinson's disease (PD). Thus, the presence or an elevated level of mSOD1 can be indicative for said diseases. Furthermore, as mentioned above, mutations in the gene encoding SOD1 can cause misfolding and account for approximately 20% of fALS cases and for a small percentage of sALS cases, but also misfolding of wt SOD1 is associated with sALS cases. Thus, the presence or an elevated level of mSOD1 is indicative for ALS.

Accordingly, the method of the present invention can be used as a method for diagnosing ALS, AD, ALS-PDC, Down's syndrome or PD, wherein the method includes assaying mSOD1 in a sample of the subject to be diagnosed, wherein the presence of mSOD1 in the sample is indicative for the above-mentioned diseases in said subject and wherein an increased level of mSOD1 in the sample compared to a control is indicative for the above mentioned diseases in said subject, respectively. In a preferred embodiment, the disease to be diagnosed with the method of the present invention is ALS.

Since misfolding of SOD1 has been observed in patients having fALS as well as in patient having sALS and since mSOD1 can be detected by the antibodies used in the method of the present invention, said method can be used to diagnose patients with with fALS and sALS.

The subject to be diagnosed may be asymptomatic or preclinical for the disease.

The method of the present invention comprises screening for mSOD1 in a sample of a patient's body fluid. The sample to be analyzed with the assay of the present invention may be any body fluid suspected to contain pathologically mSOD1, for example a blood, CSF, or urine sample. In a preferred embodiment, the sample is whole blood lysate or CSF, preferably CSF.

The method of the present invention applies an immunoassay comprising contacting the body fluid with a first anti-SOD1 antibody, wherein this first anti-SOD1 antibody is used as capture antibody. During the course of the experiments, two antibodies have been identified to be suitable for the method of the present invention, both binding to an epitope of SOD1 within the amino acid sequence 73-GGPKDEERHVGD-84 set forth in SEQ ID NO: 11. These two antibodies are designated NI-204.B and NI-204.O. As mentioned above, NI-204.B turned out to be antibody NI-204.12G7 disclosed in WO 2012/080518 A1, which binds to an epitope of SOD1 comprising the amino acid sequence 73-GGPKDEERHVG-83 as set forth in SEQ ID NO: 51 of WO 2012/080518 A1. NI-204.O binds to an epitope of SOD1 comprising the amino acid sequence 76-KDEERHVGD-84 (SEQ ID NO: 13).

In principle, the capture antibody may be any antibody or antibody format recognizing the epitope 73-GGPKDEERHVGD-84 (SEQ ID NO: 11) and preferably an epitope comprising the amino acid sequence 73-GGPKDEERHVG-83 (SEQ ID NO: 12) and/or an epitope comprising the amino acid sequence 76-KDEERHVGD-84 (SEQ ID NO: 13). In principle, such antibody may be raised against a corresponding antigen in mice, rabbits, goats, or other animal commonly used for producing polyclonal or monoclonal antibodies or by screening Fv, Fab or complete IgG libraries. Preferably, the capture antibody is a monoclonal antibody or derived from a monoclonal antibody.

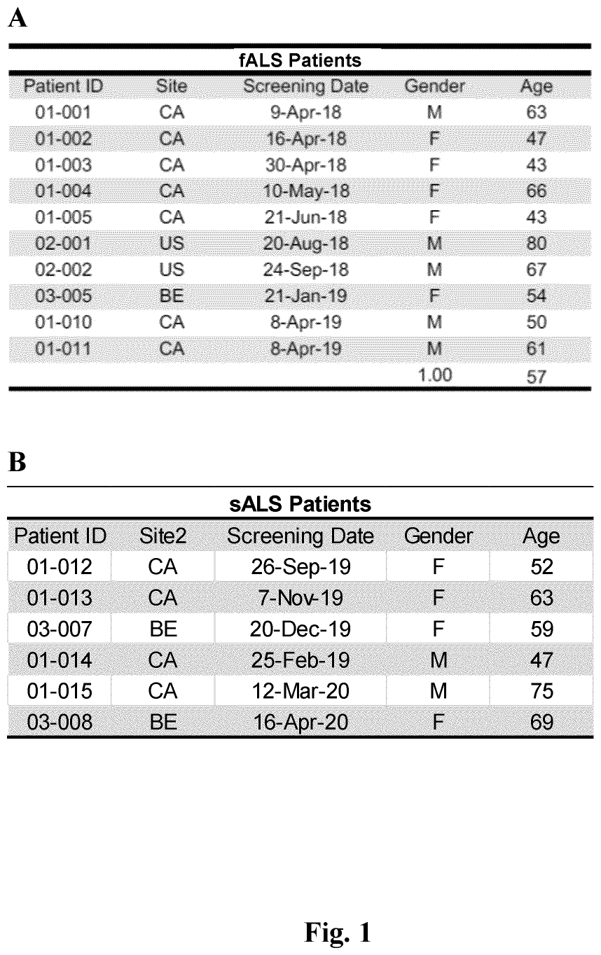

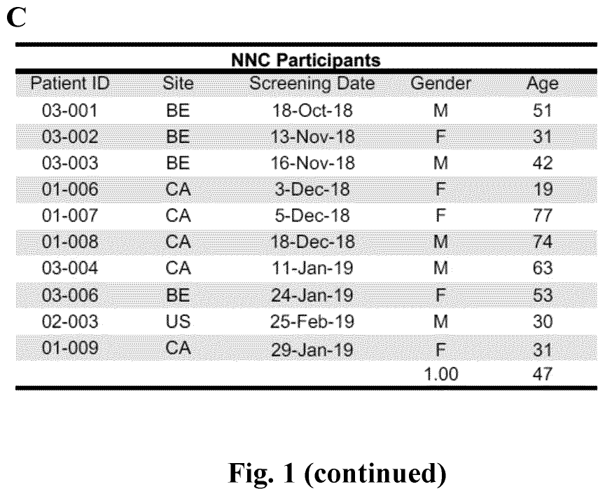

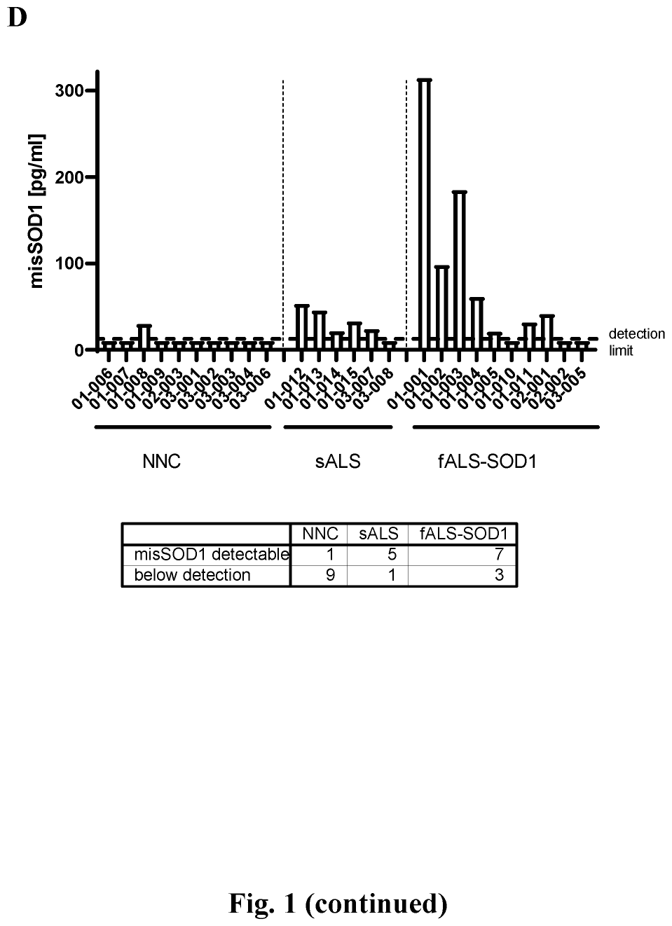

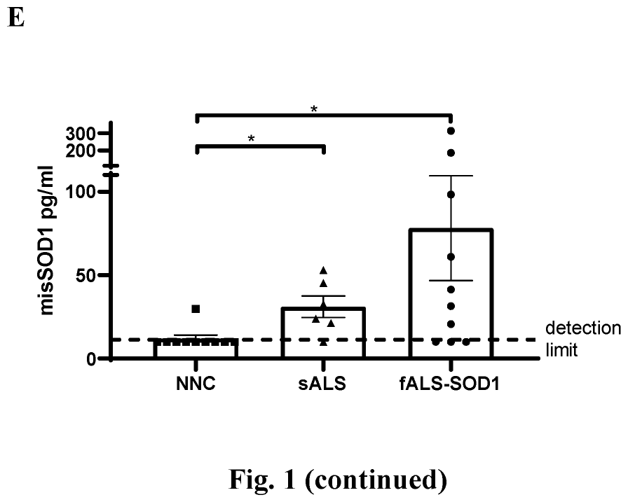

In a particular preferred embodiment of the present invention the capture antibody is derived from human antibody NI-204.12G7 and characterized by comprising in its variable region, i.e. binding domain the complementarity determining regions (CDRs) of the variable heavy (V_{H}) and variable light (V_{L}) chain having the amino acid sequences depicted in FIG. 1B of WO 2012/080518 A1, or wherein one or more of the CDRs may differ in their amino acid sequence from those set forth in FIG. 1B of WO 2012/080518 A1 by one, two, three or even more amino acids in case of CDR2 and CDR3, and wherein the capture antibody displays substantially the same or identical immunological characteristics of anti-SOD1 antibody NI-204.12G7 illustrated in the Examples of WO 2012/080518 A1. The positions of the CDRs are shown in FIG. 1B and explained in the Figure legend to FIG. 1 in WO 2012/080518 A1. The corresponding nucleotide sequences are set forth in Table II at page 54 of WO 2012/080518 A1. In addition, or alternatively, the framework regions or complete V_{H} and/or V_{L} chain are 80% identical to the framework regions depicted in FIG. 1B of WO 2012/080518 A1, preferably 85%, 90%, 95%, 96%, 97%, 98%, 99% or 100% identical to the framework regions and V_{H} and/or V_{L} chain, respectively, depicted in FIG. 1B of WO 2012/080518 A1. Furthermore, cloning and expression of antibody NI-204.B has been performed as described in WO 2012/080518 A1 in the section “Material and methods” at pages 84 to 88 which methods are thus incorporated herein by reference.

In a particular preferred embodiment, the capture antibody is characterized by the V_{H} and/or V_{L} chain depicted in FIG. 1B of WO 2012/080518 A1.

Thus, the capture antibody preferably comprises
(i) a variable heavy (V_{H}) chain comprising VH complementary determining regions (CDRs) 1, 2, and 3, and/or a variable light (V_{L}) chain comprising VL CDRs 1, 2, and 3, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 1 or 2 or a variant thereof, wherein the variant comprises one or more amino acid substitutions; and
   (b) the VL chain comprises the amino acid sequence depicted in SEQ ID NO: 6 or 7, or a variant thereof, wherein the variant comprises one or more amino acid substitutions;
   preferably wherein the VH and VL chain amino acid sequence is at least 90% identical to SEQ ID NO: 1 or 2 and 6 or 7, respectively.

In principle, the capture antibody may be any format recognizing the epitope comprising, for example chimeric antibody, single-chain antibody, Fab-fragment, bi-specific antibody, fusion antibody, labeled antibody or an analog of any one of those. Corresponding methods for producing such variants are known to the person skilled in the art and are described, e.g., in Harlow and Lane “Antibodies, A Laboratory Manual”, CSH Press, Cold Spring Harbor (1988) First edition; Second edition by Edward A. Greenfield, Dana-Farber Cancer Institute © 2014, ISBN 978-1-936113-81-1. For example, Fab and F(ab′)2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Such fragments are sufficient for use, for example, in immunodiagnostic procedures involving coupling the immunospecific portions of immunoglobulins to detecting reagents such as radioisotopes. Preferably, the capture antibody has an IgG format, i.e. being a full IgG antibody. Recombinant expression of complete human IgG1 antibodies with a human or mouse constant domain can be performed substantially as described in the Examples of WO 2012/080518 A1.

Typically, the method of the present invention further comprises the use of a second antibody as detection antibody. This antibody might be any anti-SOD1 antibody that binds to mSOD1 at an epitope different from the epitope of the capture antibody 73-GGPKDEERHVGD-84 (SEQ ID NO: 11), for example a commercially available antibody such as polyclonal rabbit anti-human SOD1 (Abcam ab52950), rabbit monoclonal anti-human SOD1 (Abcam ab79390) in combination with polyclonal biotinylated-goat anti-rabbit IgG (Jackson Immuno. 111-065-144), see, e.g., Gill et al. (2019), supra, or an anti-SOD1 antibody disclosed in WO 2012/080518 A1 or described in Tokuda et al. (2019) listed in Table 3.

In one embodiment the detection antibody is commercially available (Abcam ab185125) and is a rabbit monoclonal antibody [EPR1726] that has been raised against a synthetic SOD1^{aa 50-150} peptide and is BSA and Azide free. The antibody ab185125 is the carrier-free version of ab79390 and designed for use in antibody labeling, including fluorochromes, metal isotopes, oligonucleotides, and enzymes. Preliminary epitope analysis suggest that antibody EPR1726 binds an epitope in the same loop as the epitope of NI-204.12G7, just before the NI-204.12G7 epitope, i.e. about amino acid (61 weak) 65-75 of human SOD1. Accordingly, preferably the detection antibody for use in the method of the present invention is an antibody that shows binding characteristics similar to those of antibody EPR1726, i.e. an equivalent monoclonal antibody that binds within amino acids 50-150 of human SOD1, and in particular to amino acids (61)65-75 of human SOD1. The skilled person is well aware of means and methods how to arrive at such an equivalent antibody; see, e.g., Harlow and Lane (1988) and Greenfield (2014), Antibodies: A Laboratory Manual, supra.

Thus, preferably the detection antibody differs from the first antibody and binds to a different epitope than the capture antibody. Thus, as second antibody an antibody is used that does not compete with the first antibody for binding to mSOD1. In principle, the detection antibody may also be any format recognizing mSOD1, the second antibody is a monoclonal antibody.

In this context, during the experiments performed in accordance with the present invention it turned out that one candidate among the subset of different blinded anti-mSOD1 antibodies, designated NI-204.G while not suitable as a capture antibody could serve as a suitable second antibody, i.e. detection antibody since NI-204.G is capable of binding mSOD1 in the presence of antibody NI-204.B. Decoding the antibody probe revealed that NI-204.G corresponds to antibody NI-204.12G3 disclosed in WO 2012/080518 A1, which binds to an epitope of SOD1 comprising the amino acid sequence 121-HEKADDLGKGGNEES-135 as set forth in SEQ ID NO: 55 of WO 2012/080518 A1. Accordingly, in one embodiment the detection antibody recognizes the epitope 121-HEKADDLGKGGNEES-135 (SEQ ID NO: 14) and may be of any source and antibody format as described for the capture antibody. Preferably, the detection antibody is derived from human antibody NI-204.12G3 and characterized by comprising in its variable region, i.e. binding domain the CDRs of the V_{H} and V_{L} chain having the amino acid sequences depicted in FIG. 1H of WO 2012/080518 A1, or wherein one or more of the CDRs may differ in their amino acid sequence from those set forth in FIG. 1H of WO 2012/080518 A1 by one, two, three or even more amino acids in case of CDR2 and CDR3, and wherein the capture antibody displays substantially the same or identical immunological characteristics of anti-SOD1 antibody NI-204.12G3 illustrated in the Examples of WO 2012/080518 A1. The positions of the CDRs are shown in FIG. 1H and explained in the Figure legend to FIG. 1 in WO 2012/080518 A1. In addition, or alternatively, the framework regions or complete V_{H} and/or V_{L} chain are 80%, 85%, 90%, 95%, 96%, 97%, 98%, 99% or 100% identical to the framework regions and V_{H} and/or V_{L} chain, respectively, depicted in FIG. 1H of WO 2012/080518 A1.

However, as mentioned above, different anti-SOD1 antibodies may be useful as detection antibody as well, in particular antibodies recognizing substantially the same epitope and amino acid region recognized by antibody Abcam ab185125, Abcam ab79390 and NI-204.12G3, respectively, preferably an epitope within SOD1^{aa 100-150.} Typically, such detection antibody for use in accordance with the assay of the present invention competes with antibody Abcam ab185125, Abcam ab79390 and/or NI-204.12G3 for binding SOD1 in the sandwich ELISA format of the present invention.

Preferably, the detection antibody has an IgG format, i.e. being a full IgG antibody. Recombinant expression of complete human IgG1 antibodies with a human or mouse constant domain can be performed substantially as described in the Examples of WO 2012/080518 A1.

In one embodiment of the mSOD1 assay of the present invention, the detection antibody comprises
(i) a variable heavy (V_{H}) chain comprising VH complementary determining regions (CDRs) 1, 2, and 3, and/or a variable light (V_{L}) chain comprising VL CDRs 1, 2, and 3, wherein
   (a) VH-CDR1 comprises the amino acid sequence of SEQ ID NO: 16 or a variant thereof, wherein the variant comprises one or two amino acid substitutions,
   (b) VH-CDR2 comprises the amino acid sequence of SEQ ID NO: 17 or a variant thereof, wherein the variant comprises one or two amino acid substitutions,
   (c) VH-CDR3 comprises the amino acid sequence of SEQ ID NO: 18 or a variant thereof, wherein the variant comprises one or two amino acid substitutions,
   (d) VL-CDR1 comprises the amino acid sequence of SEQ ID NO: 20 or a variant thereof, wherein the variant comprises one or two amino acid substitutions,
   (e) VL-CDR2 comprises the amino acid sequence of SEQ ID NO: 21 or a variant thereof, wherein the variant comprises one or two amino acid substitutions, and
   (f) VL-CDR3 comprises the amino acid sequence of SEQ ID NO: 22 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 15 or a variant thereof, wherein the variant comprises one or more amino acid substitutions; and
   (b) the VL chain comprises the amino acid sequence depicted in SEQ ID NO: 19, or a variant thereof, wherein the variant comprises one or more amino acid substitutions;
      preferably wherein the VH and VL chain amino acid sequence is at least 90% identical to SEQ ID NO: 15 and 19, respectively.

In a preferred embodiment of the method of the present invention, the second antibody or a fragment thereof comprises a detectable label (e.g., a fluorescent, chemiluminescent, radioactive, enzyme, nuclear magnetic, heavy metal, a tag, a flag and the like); see, e.g., Antibodies A Laboratory Manual 2nd edition, 2014 by Cold Spring Harbor Laboratory Press, Cold Spring Harbor, N.Y., USA for general techniques; Dean and Palmer, Nat. Chem. Biol. 10 (2014), 512-523, for advances in fluorescence labeling strategies for dynamic cellular imaging; and Falck and Müller, Antibodies 7 (2018), 4; doi:10.3390/antib7010004 for enzyme-based labeling strategies for antibody-drug conjugates and antibody mimetics.

The label is either a label which can be directly detected, e.g., a fluorescent label (physicochemical reporter) or the label can be a ligand, e.g. biotin which is bound by a ligand-binding partner that comprises the directly detectable label.

In a preferred embodiment of the method of the present invention, the second antibody is conjugated to a ligand which is capable to bind a ligand-binding partner, i.e. a ligand binding tag forming a non-covalent protein-ligand interaction.

In accordance with the invention, the ligand is a moiety known to the person skilled in the art and includes affinity tags, e.g., His-Tag, maltose-binding protein (MBP)-Tag, glutathione-S-transferase (GST)-Tag, chitin binding domain or thioredoxin, calmodulin binding peptide (CBP), FLAG-peptide, Arg-Tag, Hat-Tag, c-myc-tag, S-tag, or streptavidin binding tags, e.g., Twin-Strep-Tag®, etc. as well as biotin. An overview is given for example in Terpe, Appl. Microbiol. Biotechnol. 60 (2003), 523-533 and exemplarily tags including their amino acid sequences are listed in Table 2 of Terpe (2003), which are incorporated herein by reference.

In a preferred embodiment, the ligand is or comprises biotin or a biotin analog or derivative thereof, i.e. the second antibody used in the method of the present invention is biotinylated. Biotinylation of antibodies is commonly known in the art and commercial kits are available allowing a person skilled in the art to generate a biotinylated antibody.

The method of the present invention thus comprises a labeling step with a ligand-binding partner compatible to the above-mentioned ligands. Those ligand-binding partners are also know in the art and are summarized for example in Terpe, Appl Microbiol Biotechnol 60 (2003), 523-533.

In a preferred embodiment, the ligand-binding partner is streptavidin, avidin, a streptavidin analog or an avidin analog that binds to the respective biotin or derivative. The ligand-binding partner is comprised in a conjugate which further comprises a detectable label which can be a detectable label as specified above. Preferably, the detectable label comprised in the conjugate is a chromogenic/fluorogenic or chemiluminescent label, i.e. an enzyme that is capable of catalyzing the conversion of a chromogenic/fluorogenic/chemiluminescent substrate. In a preferred embodiment, the detectable label is horseradish peroxidase or β-galactosidase. Thus, the conjugate is a streptavidin-HRP conjugate or a streptavidin-β-galactosidase (SβG) conjugate. These enzymes produce a signal when an appropriate substrate solution is added. In case of HRP the chromogenic substrate 3,3′,5,5′-tetramethylbenzidine (TMB) or 2,2′-azino-di-[3-ethylbenzthiazoline-6-sulfonic acid] (ABTS) is used or luminol or a luminol comprising substrate as a chemiluminescent substrate, and in case of β-galactosidase resorufin β-D-galactopyranoside (RGP) is used.

The method of the present invention comprises preferably a singleplex assay meaning that only one target is detected. In the method of the present invention, mSOD1 is detected as single target. However, the assay can also be designed as multiplex assay.

Thus, the method of the present invention comprises at least the following steps: Capturing mSOD1 within a sample of a subject with a first anti-SOD1 antibody attached to a surface, adding a second anti-SOD1 antibody comprising a detectable label allowing binding to mSOD1, detecting the signal of the label and comparing the signal of the label to a control.

In one embodiment, the method comprises the following steps; see also Example 1: First of all, a microplate is provided to which the first anti-SOD1 antibody as described above is spotted. Such a plate can be produced by Aushon BioSystems and is commercially available. Furthermore, the design of microarray immunoassays is generally summarized in Kusnezow et al., Mol. Cell Proteomics 5 (2006), 1681-1696. Preferably, this plate is a 96-well plate.

A further step comprises the addition of the sample comprising the body fluid to the wells of the microplate. The body fluid can be any body fluid as described above, but preferably CSF. The body fluid can also be further modified, for example purified to get rid of unwanted components or components that might interfere with the immunoassay. The sample is incubated in the wells under conditions enabling the formation of an antibody-antigen complexes, i.e. enabling binding of the first anti-SOD1 antibody to mSOD1 if present in the sample. The identification of suitable conditions can be performed by testing a positive control either simultaneously with the actual assay or beforehand to adjust the correct parameters. The positive control can be either purified mSOD1 or a sample of a patient having mSOD1 associated ALS. In a preferred embodiment, the incubation is performed for 2 h at room temperature on a plate shaker, preferably set to 600 rpm. In a preferred embodiment, the plate is washed afterwards in order to remove the unbound components.

A next step comprises the addition of the second anti-SOD1 antibody to the sample, wherein the second antibody is conjugated to a ligand. The second anti-SOD1 antibody can be an antibody or binding fragment as described above, preferably an antibody binding to a different binding site of mSOD1 and to a different epitope of mSOD1, respectively. As mentioned above, the ligand is either a label which can be directly detected, e.g., a fluorescent label or the ligand comprises a label which is bound by a ligand-binding partner that comprises the directly detectable label. In a preferred embodiment, the second antibody is biotinylated, i.e. conjugated to biotin or a biotin analog or derivative thereof.

The mixture is incubated in the wells under conditions enabling the formation a further antibody-antigen complexes, i.e. enabling binding of the second anti-SOD1 antibody to mSOD1 if present in the sample. The conditions have to be chosen such that both antibodies, i.e. the first and the second one are capable of binding mSOD1. As mentioned above, appropriate conditions can be tested with a positive control. In preferred embodiment, the incubation is performed for 30 min at room temperature on a plate shaker, preferably set to 600 rpm. In a preferred embodiment, the plate is washed afterwards in order to remove excess detection antibody.

The sample and the second antibody can also be added simultaneously to the microplate coated with the first antibody and incubation can be performed enabling binding of the first anti-SOD1 antibody to mSOD1 and of the second anti-SOD1 antibody to mSOD1.

In case the second antibody is directly labelled with a detectable label, e.g. a fluorescent label or an enzyme, an appropriate substrate solution is added.

In case indirect labeling is applied, a conjugate is added comprising a ligand-binding partner as well as a detectable label. Incubation of the sample with the conjugate in performed, preferably for further 30 min at room temperature on a plate shaker, preferably set to 600 rpm. The ligand-binding partner comprised in the conjugate can be for example streptavidin or avidin or functional analogs or derivatives thereof. In a preferred embodiment, the ligand-binding partner is streptavidin or a functionally analog or derivative thereof. The detectable label comprised in the conjugate can be any detectable label as mentioned above, but preferably it is an enzyme that is capable of catalyzing the conversion of a chromogenic/fluorogenic or chemiluminescent substrate. More preferably, the enzyme is horseradish peroxidase (HRP). Thus, the conjugate is a streptavidin-HRP reagent. Preferably a washing step is performed and afterwards, an appropriate substrate solution is added, preferably a chromogenic or chemiluminescent substrate solution. In a preferred embodiment, TMB, luminol, or a luminol comprising substrate is used.

The signal derived from the mixture is imaged. In principle, every commercial imaging system which is in particular capable of detecting and measuring fluorescence or chemiluminescence signals with high sensitivity can be used. Preferably, imaging is performed with the Cirascan™ Imaging System.

A signal from each well is detected and measured and compared to a control. Preferably the control is assayed in the same microplate within separate wells.

A control can be a reference standard, i.e. mSOD1. Alternatively, or in addition as a second control a sample of a control subject which does not have a neurodegenerative disease is used, wherein a difference between the level of mSOD1 in the sample and the control indicates that the subject to be diagnosed has a neurodegenerative disease. In particular, an elevated level of mSOD1 in the sample in comparison to said control sample is indicative for the disease, in particular for ALS. Preferably, the subject to be diagnosed and the control subject(s) are age-matched.

In one embodiment, the standard comprises a serial dilution of misfolded SOD1 from 200 ng/mL to 3 pg/mL.

In one embodiment, the microplates are covered with a lid, preferably with a lid having a fluid-absorbing matrix filled with a fluid to avoid sample evaporation during the incubation steps. The washing steps described above can be performed with any suitable buffer which does not disrupt the binding of the first antibody to the surface, the binding of the first and second antibody to mSOD1 and the binding of the conjugate to the second antibody. Preferably, washing is performed with the washing buffer of Aushon Biosciences provided in the kit as described in the Examples. As mentioned above, incubation is performed between the different steps of the assay to enable binding of the antibodies to mSOD1 and of the conjugated to the ligand of the second antibody. Of course, different incubation times can be chosen as long as binding of the antibodies and the conjugate is assured.

In another embodiment, the method of the present invention utilizes Single Molecule Arrays (Simoa™), also known as digital ELISA. In this approach, the target protein is captured on antibody-coated, paramagnetic beads, the captured proteins are labeled with an enzyme label, and single beads are isolated and sealed in arrays of femtoliter wells in the presence of enzyme substrate. The sealing step confines the fluorescent product of the enzyme-substrate reaction to ˜40 fL volume, and within 30 s the fluorescence generated by a single enzyme can be detected on an uncooled CCD camera using a white light excitation source; see Quanterix Whitepaper 6.0 (2015) with references cited therein, e.g., Rissin et al., Measurement of single protein molecules using digital ELISA. In: Wild, D. (Ed.), The Immunoassay Handbook: Theory and Applications of Ligand Binding, ELISA and Related Techniques, 4th ed. Elsevier, Oxford, UK and Rivnak et al., A fully-automated, six-plex single molecule immunoassay for measuring cytokines in blood, J. Immunol. Methods, 2015; 424:20. For example, capture beads, preferably paramagnetic beads having a diameter of about 2.7 to which surface the first antibody or binding fragment thereof as defined above is attached can be used for the method of the present invention; see Example 3. The use of such beads allows detection of mSOD1 on a single-molecule level. The sample comprising the body fluid as defined above is added to the capture beads and incubation is performed allowing capturing of mSOD1 if present in the body fluid by the beads mediated by the first anti-SOD1 antibody. As mentioned above, incubation conditions can vary and optimal conditions can be tested with a positive control. Preferably, the incubation time in 30 min. Afterwards, the second anti-SOD1 antibody which is conjugated to a ligand as defined above is added and incubation is performed allowing binding of the second anti-SOD1 antibody to the captured misfolded SOD1 on the beads. Preferably, incubation is performed for 5 min. Alternatively, the two steps as described above can be combined in that the sample and the second antibody are added to the capture beads and incubation is performed allowing capturing of misfolded SOD1 present in the body fluid by the beads mediated by the first anti-SOD1 antibody and binding of the second anti-SOD1 antibody to the captured misfolded SOD1 on the beads. Incubation time is preferably increased when combining both steps, preferably to 35 min. In case the second antibody is not directly labeled with a detectable label, but with a ligand, a conjugate as defined above is added comprising a ligand-binding partner and a detectable label, preferably wherein the ligand-binding partner is streptavidin or a functionally analog or derivative thereof and wherein the detectable label is an enzyme that is capable of developing a chromogenic or fluorescent substrate, preferably wherein the enzyme is β-galactosidase. Incubation is performed, preferably for 5 min. As also mentioned above, a fluorogenic substrate solution as defined above is added in which the beads are resuspended. Preferably, the substrate is resorufin β-D-galactopyranoside (RGP). In a next step, the beads are loaded into femtoliter-sized wells of a microplate configured to hold no more than one bead per well. Preferably, the wells have a width of about 4.25 μm and a depth of about 3.25 μm. Sealing of the wells, preferably with oil and imaging of the fluorescence signal is performed. In principle every commercially available imaging system can be used which detects signals with high sensitivity. This assay is based on the commercially available Simoa® Assay from Quanterix and thus, reagents and the Simoa™ optical system are used for said immunoassay. As already mentioned above, washing steps can be performed between the different steps of the assay.

Accordingly, in one embodiment of the method of the present invention the second anti-SOD1 antibody is conjugated to a ligand and the method comprises a labelling step with a ligand-binding tag, preferably wherein the method utilizes a Single Molecule Arrays (Simoa™) assay and optionally comprises one or more, preferably all of the following steps:
(i) attachment of the first anti-SOD1 antibody to the surface of capture beads, preferably wherein the beads are paramagnetic beads and/or have a diameter of about 2.7 μM; and
(ii)(a) addition of the sample comprising the body fluid, preferably CSF, and incubation of the beads with the sample, thereby allowing capturing of misfolded SOD1 present in the body fluid by the beads mediated by the first anti-SOD1 antibody, preferably wherein the incubation time is 30 min; and
(ii)(b) addition of the second anti-SOD1 antibody, which is conjugated to a ligand, preferably biotin or a biotin analog or derivative thereof, and incubation, thereby allowing binding of the second anti-SOD1 antibody to the captured misfolded SOD1 on the beads, preferably wherein the incubation time is 5 min; and
(ii)(c) addition of a conjugate comprising a ligand-binding tag, preferably streptavidin or a functionally analog or derivative thereof, and a detectable label, preferably an enzyme that is capable of developing a chromogenic substrate or fluorescent molecule, preferably wherein the enzyme is β-galactosidase (streptavidin-β-galactosidase (513G) conjugate), and incubation, preferably wherein the incubation time is 5 min; or
(II)(A) addition of the sample comprising the body fluid, preferably CSF, addition of the second anti-SOD1 antibody, which is conjugated to a ligand, preferably biotin or a biotin analog or derivative thereof, and incubation of the beads with the sample and the second antibody, thereby allowing capturing of misfolded SOD1 present in the body fluid by the beads mediated by the first anti-SOD1 antibody and binding of the second anti-SOD1 antibody to the captured misfolded SOD1 on the beads, preferably wherein the incubation time in 35 min; and
(II)(B) addition of a conjugate comprising the ligand-binding tag, preferably streptavidin or a functionally analog or derivative thereof, and a detectable label, preferably an enzyme that is capable of developing a chromogenic substrate or fluorescent molecule, preferably wherein the enzyme is β-galactosidase (streptavidin-β-galactosidase (513G) conjugate), and incubation, preferably wherein the incubation time is 5 min; and
(iii) resuspension of the beads in a fluorogenic substrate solution, preferably wherein the fluorogenic substrate is resorufin β-D-galactopyranoside (RGP); and
(iv) loading the beads of step (iii) into arrays of femtoliter-sized wells configured to hold no more than one bead per well; and
(v) sealing of the individual beads within the femtoliter-sized wells, preferably wherein the sealing is performed with oil; and
(vi) imaging the fluorescence signal, preferably wherein imaging is performed by the Simoa™ optical system; optionally
(vii) comparing the assayed level of misfolded SOD1 to a reference standard and/or a control.

The steps (ii)(a), (ii)(b) and (ii)(c) refer to a “three-step-approach” and the steps (II)(a) and (II)(b) refer to a “two-step-approach”.

As mentioned above, a control can be a reference standard, i.e. mSOD1. Alternatively, or in addition as a second control a sample of a control subject which does not have a neurodegenerative disease is used, wherein a difference between the level of mSOD1 in the sample and the control indicates that the subject to be diagnosed has a neurodegenerative disease. In particular, an elevated level of mSOD1 in the sample in comparison to said control sample is indicative for the disease, in particular for ALS. Preferably, the subject to be diagnosed and the control subject(s) are age-matched.

In one embodiment, the standard comprises a serial dilution of misfolded SOD1 from about 1000 ng/mL to an 8-point calibration curve by 4-fold serial dilutions down to 0.244 ng/mL, from 10 ng/mL to an 8-point calibration curve by 2-fold serial dilutions down to 0.020 ng/mL, from 50 ng/mL to a 12-point calibration curve by 2-fold serial dilutions down to 0.012 ng/mL and/or from about 66.66667 ng/mL to a 12-point calibration curve by 3-fold serial dilutions down to 0.00339 ng/mL

The method according to the present invention is highly sensitive enabling the detection of smallest amounts of mSOD1 in CSF of a subject. The high sensitivity can be especially attributed to the antibody used in the method of the present invention as first capture antibody. Dependent on the assay, mSOD1 can be detected to 6 to 7 pg/mL with acceptable precision. As regards the assay using reagents and instruments of Aushon BioSystems, more precise results were obtained within an mSOD1 range from 10.16 pg/mL to 7404.41 pg/mL leading to a reportable range between 20.32 to 14814.82 pg/mL after a 1:2 MRD (minimum required dilution). As regards the assay using reagents and instruments of from Quanterix, the assay has been shown to have an LLOD in the range of 6 pg/mL to 32 pg/ml and an LLOQ in the range of 58 pg/mL to 132 pg/mL based on a 2× assay background method and an LLOD in the range of 10.8 to 13.6 pg/mL, respectively, dependent on the capture antibody used as described in Example 3.

Thus, the method of the present invention preferably has a lower limit of quantification (LLOQ) for mSOD1 of about ≤20.32 pg/mL and a lower limit of detection (LLOD) of about 7 pg/mL, or a LLOQ of about 58 pg/mL and a LLOD of about 6 or 10 pg/mL.

Further single-molecule sensing platforms that may be employed in accordance with the method of the present invention are known to the person skilled in the art and are being developed such as low-background-noise fluorescent microscopy as well as plasmonic and electrical nanotransducers; see for review, e.g., Macchia et al., Analytical and Bioanalytical Chemistry 412 (2020), 5005-5014.

Considering the capacity to assay accessible fluids, and also the desire to have biomarkers that are confirmed in multiple studies, it would of course be a useful approach to obtain an overall picture of disease progress in any given patient, and to combine the immunoassay of the present invention with the assessment of other biomarker candidate molecules for ALS. For example, Vijayakumar et al. (2019), supra, suggest be to combine biomarker candidate molecules from across those listed in their Table 2, wherein a panel of Cystatin C, pNFH and NFL, all reflecting neuronal survival, MCP1 as a pro-inflammatory marker, the MiRs 206 and 133b reflecting muscle origin and neuromuscular junction, respectively, and some indicators of dysregulated metabolism such as homocysteine, glutamate, or cholesterol. Preferably, the immunoassay of the present invention is combined with the assessment of one or more biomarkers fluid-based biomarkers for ALS listed in Tables 1 to 5 disclosed in Vu and Bowser (2017), supra. Thus, the immunoassay of the present invention may aid in the development of a heterogeneous multi-biomarker panel for diagnostic purposes and for prognostic or predictive applications.

The present invention also encompasses therapeutic agents for use in the treatment or ameliorating the symptoms of a patient which has been diagnosed to suffer from or being at risk to develop ALS in accordance with the method of the present invention. In a preferred embodiment, the patient has been assayed to have a detectable amount of mSOD1. Preferably, the patient shows an increased level of mSOD1 when compared to a control. A control might be a healthy subject which is preferably age-matched to the patient which is diagnosed.

The patient has in a preferred embodiment at least a level of mSOD1 higher than 5 pg/mL, preferably higher than 6 or 7 pg/mL, more preferably higher than 10 pg/mL, more preferably higher than 20 pg/mL and most preferably higher than 20.32 pg/mL or 58 pg/mL.

In one embodiment, the therapeutic agent is an anti-SOD1 antibody, preferably an antibody as disclosed in WO 2012/080518 A1, preferably antibody NI-204.12G7 or an antibody as disclosed in WO 2016/120810. In another embodiment, the therapeutic agent is an agent lowering the level of SOD1, for example pyrimethamine (Lange et al. Ann. Neurol. 81 (2017), 837-848), an agent used for gene silencing, for example morpholino oligonucleotides (MOs) or an agent for unspecific treatment like rapamycin. The therapeutic agent can also be an agent used for gene therapy approaches. In a preferred embodiment, the therapeutic agent is Rilutek (riluzole) or Radicava (edavarone) both which are approved by the U.S. Food and Drug Administration for the treatment of ALS. Furthermore, the therapeutic agent can be an agent aiming at some of the specific symptoms of ALS, e.g., pain relievers or muscle relaxants. Thus, the therapeutic agent is preferably baclofen (Gablofen, Kemstro, Lioresal) or diazepam (Diastat, Valium) which can help to ease cramps. Pooling of saliva in the mouth due to difficulty in swallowing is also a symptom of ALS and can be treated with different medicines being a therapeutic agent in accordance with the present invention. Preferably, the therapeutic agent is Elavil (amitriptyline), trihexyphenidyl, Scopaderm (scopolamine patch), or Robinul (glycopyrrolate).

The present invention also encompasses a kit adapted to carry out the method of the present invention. Thus, the kit comprises the components required for performing the method of the present invention, preferably the components of the preferred embodiments of the method of the present invention.

In one embodiment, the kit is preferably suitable for use in the method of the present invention utilizing a singleplex sandwich ELISA such as the Ciraplex™ Ultrasensitive immunoassay from Aushon Biosystems illustrated in Example 1 and 2, and comprises at least a microplate which wells are pre-spotted with the first anti-SOD1 antibody as defined above, preferably including the lid as defined above. The kit further comprises a detection reagent comprising the second anti-SOD1 antibody as defined above. In addition or alternatively, the kit comprises the conjugate comprising a ligand-binding partner and a detectable label as defined above, preferably an enzyme that is capable of catalyzing the conversion of a chromogenic or chemiluminescent substrate, an appropriate substrate solution, a calibrated immunoassay standard or control of mSOD1, recommendations for buffers, diluents, substrates and/or solutions as well as instructions how to perform the assay of the present invention, and/or washing and assay/sample dilution buffer appropriate for immuno-based diagnostic assays which do not interfere with the method of the present invention and which enable the antibodies to retain in their active form.

In another embodiment, the kit is preferably suitable for use in the method of the present invention utilizing the Simoa™ assay such as illustrated in Example 3 and comprises a capture reagent comprising the first anti-SOD1 antibody as defined above and a detection reagent comprising the second anti-SOD1 antibody as defined above. In an optional embodiment, the kit comprises the beads and appropriate femtoliter-sized microplates. In addition or alternatively, the kit comprises the conjugate comprising a ligand-binding partner and a detectable label as defined above, preferably an enzyme that is capable of catalyzing the conversion of a fluorogenic substrate, an appropriate substrate solution, a calibrated immunoassay standard or control of mSOD1, recommendations for microplates, buffers, diluents, substrates and/or solutions as well as instructions how to perform the assay of the present invention, and/or washing and assay/sample dilution buffer appropriate for immuno-based diagnostic assays which do not interfere with the method of the present invention and which enable the antibodies to retain in their active form.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific example which are provided herein for purposes of illustration only and is not intended to limit the scope of the invention.

### EXAMPLES

### Example 1: Establishing and Validation of an Immunoassay Specific for mSOD1

For the quantitative measurement of mSOD1 in CSF, the Ciraplex™ Human Ultrasensitive mSOD1 1-plex immunoassay kit manufactured by Aushon BioSystems was used which is a singleplex sandwich ELISA.

### Principle of the Test:

Each well of a 96-well microplate was pre-spotted by Aushon BioSystems with the capture antibody NI-204.B, a monoclonal antibody that specifically recognizes mSOD1 at an epitope within the amino acid sequence 73-GGPKDEERHVGD-84 (SEQ ID NO: 11) of human SOD1, in particular at an epitope comprising the amino acid sequence 73-GGPKDEERHVG-83 (SEQ ID NO: 12) and that captures mSOD1 from the samples of interest. After unbound proteins were washed away, the biotinylated SOD1 rabbit monoclonal detection antibody EPR1726 as available from Abcam as ab185125 or ab79390 was added which binds to a secondary site on the mSOD1 (in this case ab185125 was used). After the removal of excess detection antibody, streptavidin-horseradish peroxidase (SA-HRP) was added. HRP is an enzyme that reacts with a substrate to produce a luminescent signal that is detected by the Cirascan™ Imaging System. The intensity of the signal produced is directly proportional to the quantity of each protein in the standard or sample of interest. The intensities are expressed at Integrated Density Values (IDV) and exported into SoftMax Pro where a weighted 5-parameter algorithm is used to back calculate unknown samples using results interpolated from the corresponding standard curve.

In particular, the assay was performed as follows:

The stock solution of 20 μg/mL mSOD1 (mSOD1 solution diluted with a stabilzyme/protease inhibitor cocktail) which was stored at −70° C. was thawed. In addition, all assay components were removed from the refrigerator/freezer and stored for 30-60 min at room temperature before use. The 1× Wash Buffer was prepared by adding the entire bottle (50 mL) to 1200 mL deionized water.

During incubation steps, a MicroClime® Lid was placed on top of the 96-well microplate. Before use, it was filled with deionized (DI) water. For this, the lid was removed from packing material and positioned in a way that the filling trough (the groove around the margin of the lid) and corners are face up. Via using a syringe or multi-channel pipette, 4 mL of DI water was slowly dispensed into the filling trough on the top of the long edge of the lid. This was repeated with the filling trough on the bottom of the long edge, so the lid contained a total of 8 mL of DI water. A kim wipe was used to remove any excess water from the troughs. When ready for the incubation steps, the lid was flipped over and placed on top of the 96-well microplate.

The standards were prepared via thawing the stock and diluting 1:100 with sample diluent. The 1:100 standard was further diluted 1:3 with sample diluent to receive the top standard. The top standard was further diluted 1:3 to have a total of 11 non-zero standards. The zero standard was standard diluent only. The samples and controls were diluted 3-fold with Sample Diluent.

The mSOD1-microplate was removed from the pouch and washed 6 times with ≥300 μL of 1×Wash Buffer using the Immuno Wash 12 manual washer. Afterwards, the plate was firmly pat dry on absorbent paper. 50 μL of standards, diluted controls and diluted samples were added to the appropriate wells in duplicate, the plate was cover with the MicroClime® Lid and incubated at room temperature for 2 h on a plate shaker set to 600 rpm. The plate was washed 4 times with ≥300 μL of 1×Wash Buffer using the Immuno Wash 12 manual washer. Afterwards, the plate was firmly pat dry on absorbent paper. 50 μL of the IgG spiked Biotinylated Antibody Reagent was added to each well, the plate was covered with the MicroClime® Lid and incubated at room temperature for 30 min on a plate shaker set to 600 rpm. Afterwards, the plate was washed 4 times with ≥300 μL of 1×Wash Buffer using the Immuno Wash 12 manual washer and patted dry on absorbent paper. 50 μL of Streptavidin-HRP Reagent was added to each well, the plate was covered with the MicroClime® Lid and incubated at room temperature for 30 min on a plate shaker set to 600 rpm. Afterwards, the plate was washed 4 times with ≥300 μL of 1×Wash Buffer using the Immuno Wash 12 manual washer and patted dry on absorbent paper.

The SuperSignal® Substrate Solution was prepared, but no more than 15 min before use, preferably just prior to the last washing step. 50 μL of mixed SuperSignal® Substrate Solution was added to each well and the plate was read within 2-4 min on the Aushon Cirascan Imaging System, thereby recording the short and long exposure time for each plate. The Integrated Density Values (IDV) from Cirasoft were transferred and processed thru SoftMax Pro Protocol and the results are quantified using a weighted 5-parameter logistic curve fit.

Using this assay, mSOD1 could be quantified to 7.01 pg/mL and 6 pg/mL with acceptable precision. Even more precise results could be obtained within an mSOD1 range from 10.16 pg/mL to 7404.41 pg/mL leading to a reportable range between 20.32 to 14814.82 pg/mL after a 1:2 MRD (minimum required dilution) of the CSF sample. Thus, the assay has a lower limit of quantification (LLOQ) for mSOD1 of about ≤20.32 pg/mL and a lower limit of detection (LLOD) of about 7 pg/mL.

### Example 2: Detection of mSOD1 in CSF Samples from fALS and sALS Patients

Using the above-described assay parameters, CSF samples were analyzed. In particular, CSF samples of 10 fALS patients with different SOD1 mutations, 6 sALS patients and 10 non-neurological control (NNC) participants were screened for mSOD1; see FIGS. 1A, B and C. These control participants do not have a neurological disorder but may have other diseases. As shown in FIGS. 1D and E, mSOD1 was detected in CSF of most fALS and sALS patients, i.e. the amount of detected mSOD1 in CSF of fALS and sALS patients was higher than the amount of mSDO1 in CSF of the control patients.

### Example 3: Establishing and Validation of a Single Molecule Array (Simoa™) Assay Specific for mSOD1

In this experiment, determining mSOD1 in a body fluid of a subject was performed using the Simoa™ assay and antibody NI-204.B as capture antibody The Simoa™ assay was performed by applying the three-step approach (30 min capture, 5 mins detection, 5 min enzyme conjugate) and the Quanterix Homebrew Kit as explained above. The sample volume was 100 μl. The capture and detection antibodies as well as the antigen stock (667 g/mL) of misfolded SOD were stored at 4° C. prior to reagent preparation. The detection antibody is commercially available (Abcam ab185125). CSF samples were kept at −80° C. until analysis.

First of all, the surface of paramagnetic beads (2.7 μm diameter) were coated with the first anti-mSOD1 antibody (capture antibody). The beads typically contain approximately 250,000 attachment sites. In particular, the capture antibody was processed following the standard Quanterix Homebrew assay protocol. The capture antibody was conjugated to magnetic beads using standard 2-step 1-ethyl-3-(3-dimethylaminopropyl)carbodiimide (EDAC) coupling chemistry at 0.7 mg/mL antibody concentrations and EDC at 0.5 mg/mL.

The second anti-mSOD1 antibody (detection antibody) was biotinylated at a molar excess of 60× using the standard Quanterix Homebrew biotinylation protocol.

The capture beads were diluted with the standard beat diluent of the Homebrew Kit and 4×10⁶ beads/mL were added to the sample solution such that there are many more beads than target molecules. Incubation was performed for 30 min. Misfolded SOD1 present in the samples are thereby captured by the capture beads. The CSF sample was diluted 2× with the Generic Homebrew Sample Diluent. The beads were then washed to remove nonspecifically bound proteins, followed by mixing of 0.1 μg/mL biotinylated detection antibodies with the capture beads. For dilution of the detector antibodies, the Generic Homebrew Detector Diluent was used. This mixture was incubated for 5 min allowing binding of the detection antibodies to the captured mSOD1 on the beads.

Following a second washing step, 300 μM of a conjugate of streptavidin-β-galactosidase (SβG) (diluted with the SβG Diluent of the Homebrew Kit) was mixed with the capture beads and incubated for 5 min. SβG binds to the biotinylated detection antibodies, resulting in enzyme labeling of captured misfolded SOD1. In this manner, each bead that has captured a single protein molecule is labeled with an enzyme. Beads that do not capture a molecule remain label free.

Following a third wash, the capture beads were resuspended in a resorufin β-D-galactopyranoside (RGP) substrate solution and transferred to the Simoa Disc. This is an array of 216,000 femtoliter-sized wells that have been sized to hold no more than one bead per well (4.25 μm width, 3.25 μm depth). The wells are subsequently sealed with oil and imaged.

If misfolded SOD1 has been captured and labeled, the β-galactosidase hydrolyzes the RGP substrate into a fluorescent product that provides the signal for measurement. A single labeled misfolded SOD1 molecule results in sufficient fluorescent signal in 30 seconds to be detected and counted by the Simoa optical system (Simoa HD-1 Analyzer (Instrument ID: 2710000020 and 2710000004 STD RUO; software version of 1.5).

The protein concentration in the test sample is determined by counting the number of wells containing both a bead and fluorescent product relative to the total number of wells containing beads. As Simoa™ assay enables concentration to be determined digitally rather than by using the total analog signal, this approach to detecting single immunocomplexes has been termed digital ELISA. At low misfolded SOD1 concentration, the percentage of bead-containing wells in the array that have a positive signal is proportional to the amount of misfolded SOD1 present in the sample. At higher target concentration, when most of the bead-containing wells have one or more labeled target molecules, the total fluorescence signal is proportional to the amount of misfolded SOD1 present in the sample. The concentration of misfolded SOD1 in unknown samples is interpolated from a standard curve.

Calibration was performed with mSOD1 via generating a calibration curve by serial dilutions starting from 1 μg/mL, which was made from an intermediate mSOD1 stock (100× dilution from 667 μg/mL stock to 6.7 μg/mL). Dilution was performed with the Generic Homebrew Calibrator Diluent A and a 4 Parameter Logistic Curve fit data reduction method (4PLC, 1/y² weighted) was used. This assay has an LLOD range of 6 pg/mL to 32 pg/ml (mean LLOD: 15.7 pg/mL) and an LLOQ range of 58 pg/mL to 132 pg/mL based on a 2× assay background method.

In a further experiment, determining mSOD1 in a body fluid of a subject using the Simoa™ assay was performed with another monoclonal antibody as capture antibody that specifically recognizes mSOD1 at an epitope within the amino acid sequence 73-GGPKDEERHVGD-84 (SEQ ID NO: 11) of human SOD1, i.e. antibody NI-204.O which recognizes an epitope comprising the amino acid sequence 76-KDEERHVGD-84 (SEQ ID NO: 13). The Simoa™ assay in principle was performed as described above, but with different reagent concentrations. In particular, conjugation of the capture antibody to the paramagnetic beads has been performed at an antibody concentration of 0.5 mg/mL; 1,500,000 capture beads/mL were added to the sample solution; the detection antibody was biotinylated at a molar excess of 60×; 0.75 μg/mL of the biotinylated detection antibody was used for the assay; 75 pM SβG were used for labelling; and the CSF sample was diluted 4× with the Generic Homebrew Sample Diluent. This assay has an LLOD range of 10.8 to 13.6 pg/mL.

1. A method of assaying misfolded SOD1 in a sample comprising a body fluid of a subject, the method comprising contacting the body fluid with a first anti-SOD1 antibody which binds to an epitope of SOD1 within the amino acid sequence 73-GGPKDEERHVGD-84 set forth in SEQ ID NO: 11 as a capture antibody and a second anti-SOD1 antibody which binds to an epitope of human SOD1^{aa 50-150} as a detection antibody.

2. The method according to claim 1, wherein the body fluid is cerebrospinal fluid (CSF).

3. The method according to claim 1 or 2, wherein the presence of misfolded SOD1 is indicative for amyotrophic lateral sclerosis (ALS) in the subject.

4. A method of diagnosing ALS in a subject comprising the steps of the method according to any one of claims 1 to 3, wherein the presence or increased level of misfolded SOD1 in the sample compared to a control is indicative for ALS in said subject.

5. The method according to claim 3 or 4, wherein ALS is familial ALS (fALS).

6. The method according to claim 3 or 4, wherein ALS is sporadic ALS (sALS).

7. The method according to any one of claims 1 to 6, wherein the first antibody is a monoclonal antibody.

8. The method according to any one of claims 1 to 7, wherein the second antibody is a monoclonal antibody.

9. The method according to any one of claims 1 to 8, wherein the first antibody is characterized by comprising in its variable region, i.e. binding domain
(i) the six CDRs of the variable heavy (V_{H}) and variable light (V_{L}) chain, wherein:
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions; and/or
(ii) a VH chain and a VL chain, wherein
(a) the VH chain comprises an amino acid sequence which is at least 90% identical to the amino acid sequence depicted in SEQ ID NO: 1 or 2; and
(b) the VL chain comprises an amino acid sequence which is at least 90% identical to the amino acid sequence depicted in SEQ ID NO: 6 or 7.

10. The method according to any one of claims 1 to 9, wherein the second antibody is characterized by binding to an epitope of SOD1 within the amino acid sequence 121-HEKADDLGKGGNEES-135 set forth in SEQ ID NO: 14 and comprising in its variable region, i.e. binding domain
(i) the six CDRs of the V_{H} and V_{L} chain, wherein
(a) VH-CDR1 comprises the amino acid sequence of SEQ ID NO: 16 or a variant thereof, wherein the variant comprises one or two amino acid substitutions,
(b) VH-CDR2 comprises the amino acid sequence of SEQ ID NO: 17 or a variant thereof, wherein the variant comprises one or two amino acid substitutions,
(c) VH-CDR3 comprises the amino acid sequence of SEQ ID NO: 18 or a variant thereof, wherein the variant comprises one or two amino acid substitutions,
(d) VL-CDR1 comprises the amino acid sequence of SEQ ID NO: 20 or a variant thereof, wherein the variant comprises one or two amino acid substitutions,
(e) VL-CDR2 comprises the amino acid sequence of SEQ ID NO: 21 or a variant thereof, wherein the variant comprises one or two amino acid substitutions, and
(f) VL-CDR3 comprises the amino acid sequence of SEQ ID NO: 22 or a variant thereof, wherein the variant comprises one or two amino acid substitutions; and/or
(ii) a VH chain and a VL chain, wherein
(a) the VH chain comprises an amino acid sequence which is at least 90% identical to the amino acid sequence depicted in SEQ ID NO: 15; and
(b) the VL chain comprises an amino acid sequence which is at least 90% identical to the amino acid sequence depicted in SEQ ID NO: 19

11. The method according to any one of claims 1 to 10, wherein the second anti-SOD1 antibody is conjugated to a detectable label.

12. The method according to claim 11, wherein the detectable label is selected from the group consisting of an enzyme, a radioisotope, a fluorescent compound, a chemiluminescent compound, a bioluminescent compound, a tag, a flag, a ligand and a heavy metal.

13. The method according to claim 11 or 12, wherein the second anti-SOD1 antibody is conjugated to a ligand and wherein the method comprises a labelling step with a ligand-binding tag, wherein the method comprises the following steps:
(i) providing a microplate to which wells the first anti-SOD1 antibody is spotted; and
(ii) addition of the sample comprising the body fluid to the wells followed by incubation, thereby allowing capturing of misfolded SOD1 present in the body fluid by the first anti-SOD1 antibody, preferably wherein incubation is performed for 2 h at room temperature on a plate shaker set to 600 rpm; and
(iii) addition of the second anti-SOD1 antibody, which is conjugated to a ligand and incubation, thereby allowing binding of the second anti-SOD1 antibody to the captured misfolded SOD1, preferably wherein incubation is performed for 30 min at room temperature on a plate shaker set to 600 rpm; and
(iv) addition of a conjugate comprising a ligand-binding tag and a detectable label and incubation, preferably wherein incubation is performed for 30 min at room temperature on a plate shaker set to 600 rpm; and
(v) addition of a chromogenic or chemiluminescent substrate solution; and
(vi) imaging the signal; optionally
(vii) comparing the assayed level of misfolded SOD1 to a reference standard and/or a control, preferably wherein the reference standard and/or the control is added to the same microplate than the sample,
optionally wherein the method is a singleplex immunoassay.

14. The method according to claim 13, wherein
(i) the microplate is a 96-well plate
(ii) the body fluid is cerebrospinal fluid (CSF);
(iii) the ligand is biotin or a biotin analog or derivative thereof;
(iv) the ligand-binding tag is streptavidin or a functionally analog or derivative thereof and wherein the detectable label is an enzyme that is capable of catalyzing the conversion of a chromogenic or chemiluminescent substrate, preferably wherein the enzyme is horseradish peroxidase;
(v) the substrate is 3,3′,5,5′-tetramethylbenzidine (TMB) or a luminol substrate;
(vi) imaging is performed by the Cirascan™ Imaging System;
(vii) a washing step is performed at least after steps (ii), (iii) and/or (iv); and/or
(viii) the microplate is covered with a lid, preferably with a lid having a fluid-absorbing matrix filled with a fluid during incubation steps.

15. The method according to any one of claims 1 to 14, wherein the method has a lower limit of quantification (LLOQ) for misfolded SOD1 of ≤20.32 pg/mL.

16. The method according to any one of claims 1 to 12, utilizing Single Molecule Arrays (Simoa™), preferably comprising the steps:
(i) attachment of the first anti-SOD1 antibody to the surface of capture beads; and
(ii)(a) addition of the sample comprising the body fluid and incubation of the beads with the sample, thereby allowing capturing of misfolded SOD1 present in the body fluid by the beads mediated by the first anti-SOD1 antibody; and
(ii)(b) addition of the second anti-SOD1 antibody, which is conjugated to a ligand, and incubation, thereby allowing binding of the second anti-SOD1 antibody to the captured misfolded SOD1 on the beads; and
(ii)(c) addition of a conjugate comprising a ligand-binding tag and a detectable label, and incubation; or
(II)(A) addition of the sample comprising the body fluid, addition of the second anti-SOD1 antibody, which is conjugated to a ligand, and incubation of the beads with the sample and the second antibody, thereby allowing capturing of misfolded SOD1 present in the body fluid by the beads mediated by the first anti-SOD1 antibody and binding of the second anti-SOD1 antibody to the captured misfolded SOD1 on the beads; and
(II)(B) addition of a conjugate comprising the ligand-binding tag and a detectable label, and incubation; and
(iii) resuspension of the beads in a chromogenic/fluorogenic substrate solution; and
(iv) loading the beads of step (iii) into arrays of femtoliter-sized wells configured to hold no more than one bead per well; and
(v) sealing of the individual beads within the femtoliter-sized wells; and
(vi) imaging the signal; optionally
(vii) comparing the assayed level of misfolded SOD1 to a reference standard and/or a control.

17. The method according to claim 16, wherein
(i) the beads are paramagnetic beads and/or have a diameter of about 2.7 μM;
(ii)(a) the body fluid is CSF and/or the incubation time is 30 min;
(ii)(b) the ligand is biotin or a biotin analog or derivative thereof and/or the incubation time is 5 min;
(ii)(c) the ligand-binding tag is streptavidin or a functionally analog or derivative thereof, the detectable label is an enzyme that is capable of converting a chromogenic/fluorogenic substrate, preferably wherein the enzyme is β-galactosidase, and/or the incubation time is 5 min;
(II)(A) the body fluid is CSF, the ligand is biotin or a biotin analog or derivative thereof, and/or incubation time in 35 min;
(II)(B) the ligand-binding tag is streptavidin or a functionally analog or derivative thereof, the detectable label is an enzyme that is capable of converting a chromogenic/fluorogenic substrate, preferably wherein the enzyme is β-galactosidase, and/or incubation time is 5 min;
(iii) the fluorogenic substrate is resorufin β-D-galactopyranoside (RGP);
(v) the sealing is performed with oil;
(vi) the imaging is performed by the Simoa™ optical system.

18. Use of an anti-SOD1 antibody which binds to an epitope of SOD1 within the amino acid sequence 73-GGPKDEERHVGD-84 set forth in SEQ ID NO: 11 as a capture antibody and/or an anti-SOD1 antibody which binds to an epitope of human SOD 1^{aa 50-150} as a detection antibody in the method according to any one of claims 1 to 17 for assaying misfolded SOD1.

19. The use of claim 18, wherein the capture antibody is characterized as defined in claim 9 and/or the detection antibody is characterized as defined in claim 10.

20. A therapeutic agent for use in the treatment or ameliorating the symptoms of a patient which has been diagnosed to suffer from or being at risk to develop ALS in accordance with the method according to any one of claims 1 to 17, preferably wherein the patient has been diagnosed to suffer from or being at risk to develop sALS, preferably wherein the patient has been assayed to have a detectable amount of misfolded SOD1 and/or an increased level of misfolded SOD1 when compared to a control.

21. A kit adapted to carry out the method according to any one of claims 1 to 17 for assaying of misfolded SOD1 in a sample comprising body fluid of a subject, comprising at least
(i) a first monoclonal anti-SOD1 antibody which binds to an epitope of SOD1 within the amino acid sequence 73-GGPKDEERHVGD-84 set forth in SEQ ID NO: 11; and
(ii) a detection reagent comprising a second monoclonal anti-SOD1 antibody which binds to an epitope of human SOD1^{aa 50-150} as a detection antibody and wherein the second anti-SOD antibody is conjugated to a detectable label, preferably wherein the detectable label is selected from the group consisting of an enzyme, a radioisotope, a fluorescent compound, a chemiluminescent compound, a bioluminescent compound, a tag, a flag, a ligand and a heavy metal; and optionally
(iii) a conjugate comprising a detectable-label-binding tag and a detectable label, preferably wherein the detectable-label-binding tag is a ligand-binding tag and wherein the detectable label is an enzyme that is capable of catalyzing the conversion of a chromogenic, chemiluminescent, or fluorogenic substrate;
(iv) a chromogenic, chemiluminescent, or fluorogenic substrate solution;
(v) a calibrated immunoassay standard or control of misfolded SOD1;
(vi) recommendations for microplates, buffers, diluents, substrates and/or solutions as well as instructions how to perform the assay according to any one of claims 1 to 17; and/or
(vii) washing and assay/sample dilution buffer.

22. The kit of claim 21, wherein
(i) the first antibody is pre-spotted to the wells of a microplate, preferably a 96-well microplate including a lid, preferably wherein the first antibody is characterized as defined in claim 9;
(ii) the detectable label is a ligand, preferably biotin or a biotin analog or derivative thereof;
(iii) the ligand-binding tag is streptavidin or a functionally analog or derivative thereof, and the detectable label is an enzyme that is capable of catalyzing the conversion of a chromogenic or chemiluminescent substrate, preferably wherein the detectable label is horseradish peroxidase;
(v) the substrate solution is a chromogenic or chemiluminescent substrate solution, preferably wherein the substrate is TMB or a luminol substrate; and/or
(vi) the standard comprises a serial dilution of misfolded SOD1 from 200 ng/mL to 3 pg/mL.

23. The kit according to claim 21 or 22 comprising:
(i) a microplate, preferably a 96-well microplate including a lid, which wells are pre-spotted with a first monoclonal anti-SOD1 antibody as capture antibody which is characterized as defined in claim 9;
(ii) a second biotinylated anti-SOD1 antibody as detection antibody;
(iii) a streptavidin-HRP reagent;
(v) a substrate solution comprising TMB or luminol;
(vi) a calibrated immunoassay standard or control of misfolded SOD1; and
(vii) washing and assay/sample dilution buffer.

24. The kit of claim 21, wherein
(i) the first monoclonal anti-SOD1 antibody is comprised in a capture reagent, preferably wherein the first antibody is characterized as defined in claim 9;
(ii) the detectable label is a ligand, preferably biotin or a biotin analog or derivative thereof;
(iii) the ligand-binding tag is streptavidin or a functionally analog or derivative thereof, and the detectable label is an enzyme that is capable of converting a chromogenic/fluorogenic substrate, preferably wherein the detectable label is β-galactosidase;
(iv) the substrate solution is a chromogenic or fluorogenic substrate solution, preferably wherein the substrate is resorufin β-D-galactopyranoside (RGP); and/or
(v) the standard comprises a serial dilution of misfolded SOD1 from about 1000 ng/mL to an 8-point calibration curve by 4-fold serial dilutions down to 0.244 ng/mL, from 10 ng/mL to an 8-point calibration curve by 2-fold serial dilutions down to 0.020 ng/mL, from 50 ng/mL to a 12-point calibration curve by 2-fold serial dilutions down to 0.012 ng/mL and/or from about 66.66667 ng/mL to a 12-point calibration curve by 3-fold serial dilutions down to 0.00339 ng/mL, and optionally
wherein the kit further comprises capture beads, preferably paramagnetic beads having a diameter of about 2.7 μM.

25. The kit of claim 21 or 24 comprising:
(i) a capture reagent comprising a first monoclonal anti-SOD1 antibody as capture antibody which is characterized as defined in claim 9;
(ii) a second biotinylated anti-SOD1 antibody as detection antibody;
(iii) a streptavidin-β-galactosidase (SβG) reagent;
(iv) a substrate solution comprising RGP;
(v) a calibrated immunoassay standard or control of misfolded SOD1;
(vi) washing and assay/sample dilution buffer; and
(vii) paramagnetic capture beads having a diameter of about 2.7 μM.
